(19) United States
(12) Patent Application Publication
(10) Pub. No.: US 2010/0076276 A1
(43) Pub. Date: Mar. 25, 2010

(54) Medical Sensor, Display, and Technique For Using The Same

(75) Inventor: Bruce R. Gilland, Superior, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/237,535

(22) Filed: Sep. 25, 2008

(51) Int. Cl. A61B 5/00

(52) U.S. Cl. 600/301

(57) ABSTRACT

According to embodiments, a wearable sensor assembly may include an electronic paper display and/or a flexible gel battery. The electronic paper display may have reduced power consumption in addition to being comfortable, flexible, and lightweight. In addition, a pulse oximetry system and/or monitor may include a gel battery to facilitate rapid recharging after battery use.

### BACKGROUND

The present disclosure relates generally to medical devices and, more particularly, to sensors used for sensing physiological parameters of a patient.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

Pulse oximetry readings typically involve placement of a sensor on a patient's tissue. The sensors may be coupled to a display, such as a downstream monitor or an integral display, that allows a healthcare provider or a patient to view the information collected by the sensor and make certain determinations based on that information. For sensors that include integral displays, various challenges may arise in providing a display that is able to be read easily and that does not consume battery power so quickly that the display has only limited run time before the battery life runs out. A display that is continuously illuminated may involve using a larger battery as a power source in order to adequately light the display. However, for sensors that are meant to be worn while the patient is active, a larger battery may be cumbersome and uncomfortable for the patient.

### BRIEF DESCRIPTION OF THE DRAWINGS

Advantages of the disclosure may become apparent upon reading the following detailed description and upon reference to the drawings in which:

FIG. 1 is a perspective view of an exemplary wristband sensor assembly that includes a medical sensor and an electronic paper display;

FIG. 2 is a perspective view of an exemplary hat-based sensor assembly that includes a medical sensor and an electronic paper display;

FIG. 3 is a block diagram of an exemplary sensor assembly;

FIG. 4 is a stack diagram of an exemplary sensor assembly; and

FIG. 5 is a block diagram of an exemplary pulse oximetry system.

### DETAILED DESCRIPTION

One or more embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In an embodiment, sensors or other applications utilizing spectrophotometry are provided herein that include flexible electronic paper displays, such as electrophoretic displays. Used in conjunction with wearable medical sensors, such displays may provide multiple advantages. Electronic paper displays have a paper-like look that provides a high contrast, flicker-free display with a wide viewing angle and relative ease of readability under a wide range of lighting conditions, including low light. Because such electronic paper displays, including electrophoretic displays, are thin and relatively flexible, these displays may be incorporated into sensors that comfortably conform to a patient's tissue. An additional benefit provided by sensors that include electronic paper may be reduced power consumption because electronic paper displays only consume power when new information is being written to the display, i.e., power is not consumed to maintain information on the display. For sensors that operate remotely, such reduced power consumption may lead to increased wear times and decreased battery waste, as the batteries may be recharged less frequently. In addition, electronic paper displays, because they have relatively low power consumption, may not experience substantial temperature increases during operation, and may be more comfortable for the wearer.

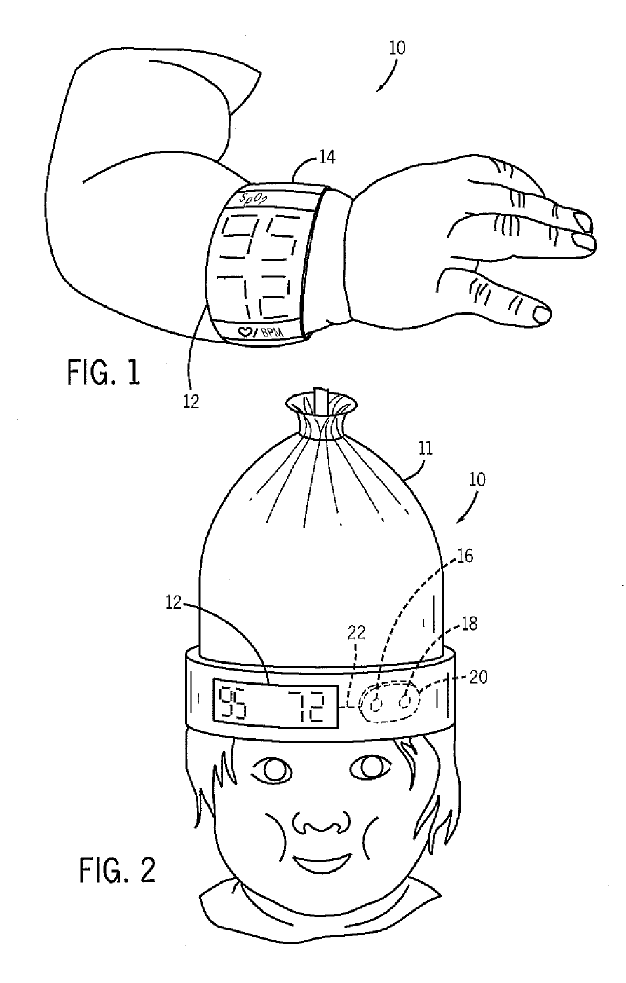

In an embodiment, an electronic paper display may be adapted for placement in a wearable medical sensor assembly such as a wristband, hat (for example, a neonatal stocking cap), a headband, or other wearable structure (i.e. a glove or a sock) to apply the sensor on the body of the user. FIG. 1 illustrates a sensor assembly 10 including a wristband structure 14 and an electronic paper display 12. The electronic paper display 12 may be capable of displaying medical parameter and/or monitoring information gathered by one or more medical sensors on or in communication with the wearable structure. The electronic paper display 12 may be incorporated into or onto the wristband structure 14. The electronic paper display 12 may be any suitable size or shape that is adapted for viewing by a patient or healthcare provider.

In an embodiment, a wearable sensor assembly 10 may include, as in FIG. 2, a reflectance-type pulse oximetry sensor 20 that may be adapted to be placed or adhered to the inside of a wearable garment, such as a hat 11. The sensor 20 may include an emitter 16 containing emitters for two or more wavelengths and a detector 18 spaced apart from the emitter 16. The signals from the detector 18 may be carried to an electronic paper display 12 by one or more leads 22. The electronic paper display 12 may be positioned on the hat 11 so that a healthcare provider may easily view the display and read the relevant information.

In the embodiment depicted in FIG. 2, the electronic paper display 12 may be capable of displaying oxygen saturation information and/or heart rate information. In other embodiment, the electronic paper display may be capable of displaying addition information derived from the data collected by the sensor 20, including trend data, alarm data, and data related to clinical conditions including sleep apnea. In embodiments, the display 12 is updated at the rate of once every half second or less frequently, such as once every second, every two seconds, three seconds, etc.

In an embodiment, sensors and/or systems or other applications utilizing spectrophotometry are provided herein that include gel batteries. Gel batteries recharge quickly compared to conventional batteries and may allow medical sensors and devices to spend less time plugged into an AC power source to recharge and, consequently, more time in use. Additionally, gel batteries may be flexible and lightweight. In an embodiment, a flexible gel battery may be incorporated into a sensor with a flexible electronic paper display to provide a relatively lightweight, conformable sensor structure that has reduced power consumption and recharges to full power relatively quickly, so that the sensor may worn for extended periods of time. In another embodiment, a standalone or multiparameter medical monitor may include a gel battery.

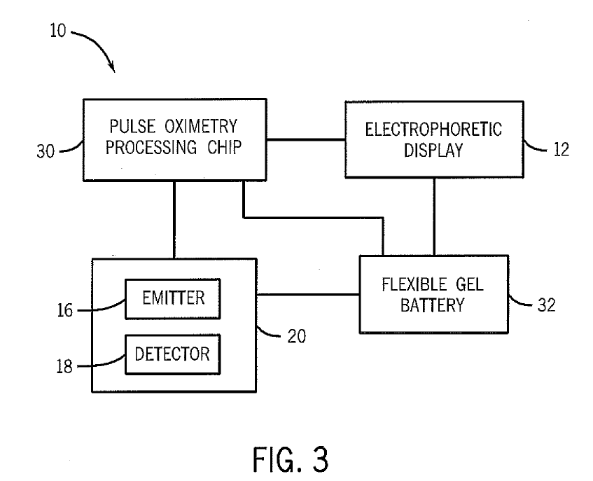

FIG. 3 is a block diagram of an exemplary sensor assembly 10 including an electronic display, shown here as an electrophoretic display 12, coupled to a sensor 20, according to an embodiment. In the depicted embodiment, the sensor 20 includes an emitter 16 and a detector 18. The sensor assembly may also include a pulse oximetry processing chip 30 for driving the emitter 16, processing the signal from the detector 18, and providing an output to the electrophoretic display 12. Also depicted is a flexible gel battery 32 that may provide power to the sensor 20, the processing chip 30, and/or the electrophoretic display 12. Activating (i.e., turning on) the sensor assembly 10 may involve driving the emitter 16 to shine light through a patient's tissue. The light that subsequently impinges the detector 18 may generate a signal that may be sent to the pulse oximetry processing chip 30, where the signal may be processed to derive an output to be displayed on the electrophoretic display 12.

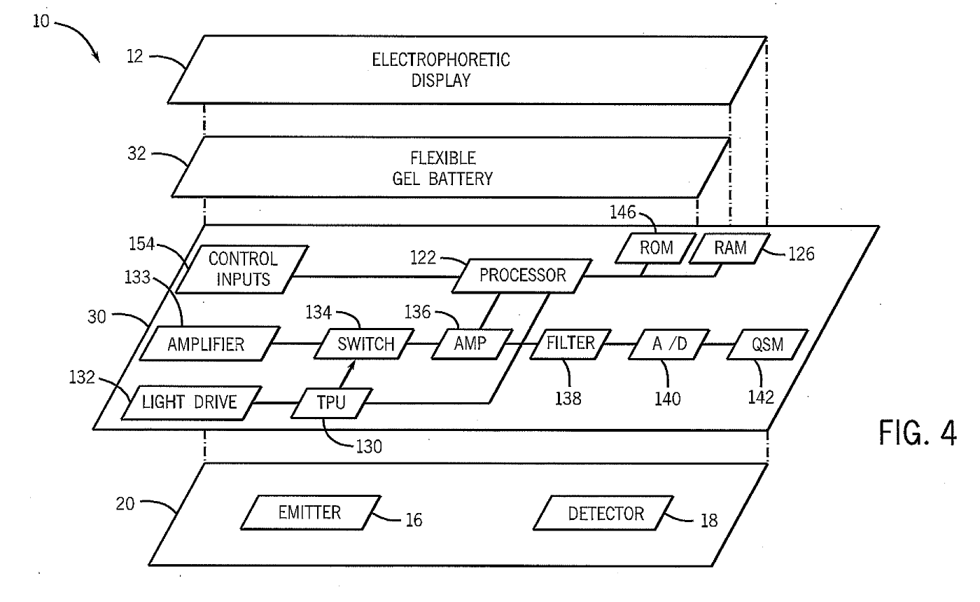

FIG. 4 is a stack diagram of the sensor assembly 10 of FIG. 3, according to an embodiment. The sensor assembly 10 includes an emitter 16 and a detector 18 that may be of any suitable type. For example, the emitter 16 may be one or more light emitting diodes adapted to transmit one or more wavelengths of light in the red to infrared range, and the detector 18 may one or more photodetectors selected to receive light in the range or ranges emitted from the emitter 16. Alternatively, an emitter 16 may also be a laser diode or a vertical cavity surface emitting laser (VCSEL). An emitter 16 and detector 18 may also include optical fiber sensing elements. An emitter 16 may include a broadband or “white light” source, in which case the detector could include any of a variety of elements for selecting specific wavelengths, such as reflective or refractive elements or interferometers. These kinds of emitters and/or detectors may be coupled to the sensor via fiber optics. Alternatively, a sensor assembly 10 may sense light detected from the tissue is at a different wavelength from the light emitted into the tissue. Such sensors may be adapted to sense fluorescence, phosphorescence, Raman scattering, Rayleigh scattering and multi-photon events or photoacoustic effects.

For pulse oximetry applications using either transmission or reflectance type sensors the oxygen saturation of the patient's arterial blood may be determined using two or more wavelengths of light, most commonly red and near infrared wavelengths. Similarly, in other applications, a tissue water fraction (or other body fluid related metric) or a concentration of one or more biochemical components in an aqueous environment may be measured using two or more wavelengths of light, most commonly near infrared wavelengths between about 1,000 nm to about 2,500 nm. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra.

The emitter 16 and the detector 18 may be disposed on a sensor body, which may be made of any suitable material, such as plastic, foam, woven material, or paper. In one embodiment, the emitter 16 and the detector 18 may be disposed on or embedded in a molded rigid polymer housing that provided a fixed optical distance between the emitter 16 and the detector 18.

In an embodiment, the sensor assembly 10 may be a “transmission type” sensor. Transmission type sensors include an emitter 16 and detector 18 that are typically placed on opposing sides of the sensor site. If the sensor site is a fingertip, for example, the sensor assembly 10 is positioned over the patient's fingertip such that the emitter 16 and detector 18 lie on either side of the patient's nail bed. In other words, the sensor assembly 10 is positioned so that the emitter 16 is located on the patient's fingernail and the detector 18 is located 180° opposite the emitter 16 on the patient's finger pad. During operation, the emitter 16 shines one or more wavelengths of light through the patient's fingertip and the light received by the detector 18 is processed to determine various physiological characteristics of the patient. In each of the embodiments discussed herein) it should be understood that the locations of the emitter 16 and the detector 18 may be exchanged. For example, the detector 18 may be located at the top of the finger and the emitter 16 may be located underneath the finger. In either arrangement, the sensor assembly 10 will perform in substantially the same manner.

Reflectance type sensors also operate by emitting light into the tissue and detecting the light that is transmitted and scattered by the tissue. However, reflectance type sensors include an emitter 16 and detector 18 that are typically placed on the same side of the sensor site. For example, a reflectance type sensor may be placed on a patient's fingertip or forehead such that the emitter 16 and detector 18 lie side-by-side. Reflectance type sensors detect light photons that are scattered back to the detector 18. A sensor assembly 10 may also be a “transflectance” sensor, such as a sensor that may subtend a portion of a baby's heel.

As shown, the emitter 16 and detector 18 may be coupled to a processing chip 30. In an embodiment, the processing chip 30 may include one or more “general-purpose” microprocessors, one or more special-purpose microprocessors and/or ASICS, or some combination thereof. The processing chip 30 may include circuitry and/or other structures that function as a RAM memory 126, a time processing unit (TPU) 130, and/or light drive circuitry 132. The TPU 130 may provide timing control signals to light drive circuitry 132, which controls when the emitter 16 is activated, and if multiple light sources are used, the multiplexed timing for the different light sources. The processing chip 30 may also provide the functionality of an amplifier 133 and a switching circuit 134. These functions of the processing chip 30 may allow signals to be sampled at the proper time, depending at least in part upon which of multiple light sources is activated, if multiple light sources are used. In addition, the processing chip 30 may provide additional amplification functions 136, low pass filtering functions 138, and/or analog-to-digital converter functions 140 to process the received signal. The digital data may then be stored in a queued serial module (QSM) 142 provided on the processing chip 30 for later downloading to RAM 126 as QSM 142 fills up. In an embodiment, there may be multiple parallel paths of separate amplifier, filter, and A/D converters for multiple light wavelengths or spectra received.

In an embodiment, based at least in part upon the received signals corresponding to the light received by detector 18, microprocessor 122 may calculate the oxygen saturation using various algorithms. These algorithms may require coefficients, which may be empirically determined and may correspond to the wavelengths of light used. The algorithms may be stored in a ROM 146 of the processing chip 30 and accessed and operated according to microprocessor 122 instructions. Furthermore, any number of methods or algorithms may be used to determine a patient's pulse rate, oxygen saturation or any other desired physiological parameter.

In an embodiment, the processing chip 30 may be coupled to one or more flexible gel batteries 32. For example, the flexible gel battery 32 may be an organic radical battery (NEC Corporation, Irving, Tex.) Organic radical batteries use an organic radical compound to produce energy. The compound used in the organic radical battery is referred to as organic radical polymer and may include a stable radical that may take the form of a gel permeated with electrolytes. The flexible gel battery may be a thin sheet, such as a 300 microns thick sheet, or may be slightly thicker than a business card. In certain embodiments, because the gel offers little electrical resistance, the flexible gel battery may be fully charged in less than 30 seconds.

In an embodiment, the flexible gel battery may also be coupled to an electrophoretic display 12, such as those available from E Ink Corporation (Cambridge, Mass.) or SiPix Imagine (Fremont, Calif.). For example, the electrophoretic display 12 may include microcapsules of electronic ink (such as the Electronic Ink available from E Ink Corporation) printed onto sheets of plastic film. This film may be laminated to a layer of electronic drive circuitry, which in turn can be addressed by a driver. The microcapsules contain small particles, suspended in fluid, which may be in different color combinations and may be positively or negatively charged. In one embodiment, white particles may be positively charged and black particles may be negatively charged. Without any electrical bias from the drive circuitry, these particles are randomly distributed within a capsule and that pixel, under reflective light, appears gray. If a positive bias is applied to a microcapsule, the white particles will move to the viewable area of the microcapsule and the black particles will migrate to the bottom of the microcapsule. The microcapsule will, therefore, appear white. Similarly, if a negative charge is applies to a microcapsule, it will appear black. In an embodiment, other combinations are possible, such as blue/white or green/white. Similarly, either color may be associated with either positively or negatively charged particles.

In one embodiment, a higher resolution display can be achieved through the use of a subcapsule addressing. Since the microcapsules are suspended in a liquid “carrier medium” they may be printed on almost any surface, including glass, plastic, fabric and even paper. In an embodiment, an electrophoretic display 12 may be coated onto many different surfaces using appropriate binders such as PVCs, urethanes and silicon binders.

In another embodiment, an electronic paper display 12 may include an electro-wetting display. Electro-wetting technology is based on controlling the shape of a confined water/oil interface by an applied voltage. With no voltage applied the (colored) oil forms a flat film between the water and a hydrophobic (water-repellent), insulating coating of an electrode, resulting in a colored pixel. When a voltage is applied between the electrode and the water, the interfacial tension between the water and the coating changes. As a result the stacked state is no longer stable, causing the water to move the oil aside. This results in a partly transparent pixel, or, in case a reflective white surface is used under the switchable element, a white pixel. Because of the small size of the pixel, the user only experiences the average reflection, which means that a high-brightness, high-contrast switchable element is obtained, which forms the basis of the reflective display. In such an embodiment, the electronic paper display 12 may have the capability of providing video content and/or a full-color display. In one embodiment, instead of using red, green and blue (RGB) filters or alternating segments of the three primary colors, which effectively result in only one third of the display reflecting light in the desired color, electro-wetting allows for a system in which one sub-pixel is able to switch two different colors independently. This results in the availability of two thirds of the display area to reflect light in any desired color by building up a pixel with a stack of two independently controllable colored oil films plus a color filter.

In another embodiment, the electronic paper display 12 may utilize bistable LCD technology (B&W and color) based polymer molecules in one of two stable states, the Uniform (U) state and the Twisted (T) state, which are selected by applying current via in-plane electrodes. Once either state is selected, it is maintained without consuming any additional power. Alternatively, a cholesteric LCD uses organic transistors embedded into flexible substrates. An array of pixels is divided into triads, typically consisting of the standard cyan, magenta and yellow, in the same way as CRT monitors (although using subtractive primary colors as opposed to additive primary colors). The display 12 is then controlled like any other electronic color display.

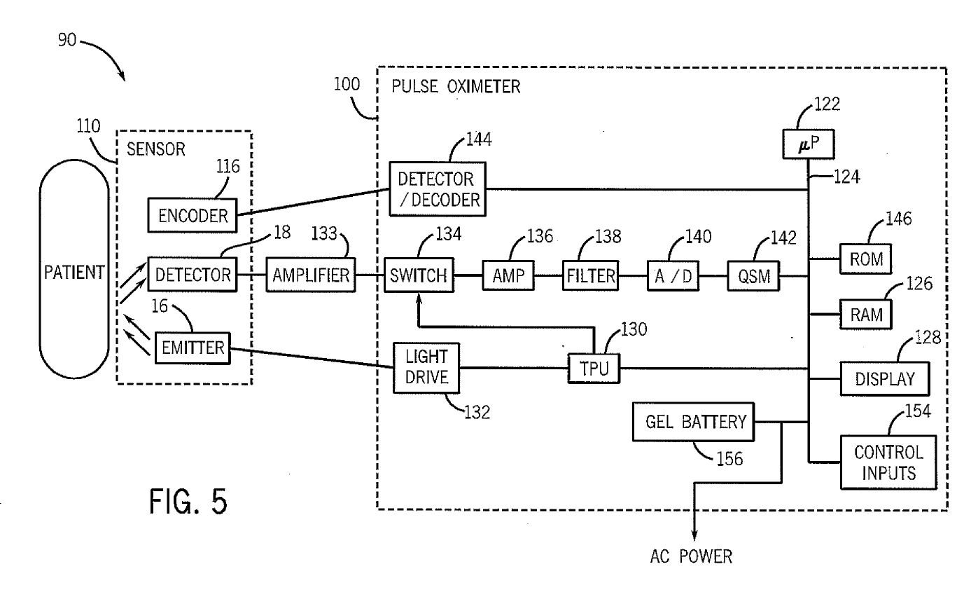

In addition to embodiments for wearable sensors that may include flexible displays and batteries, it is envisioned that electronic paper displays and/or gel batteries may also be incorporated into conventional standalone monitors or multiparameter monitors that work with conventional disposable or reusable medical sensors. FIG. 5 is a block diagram of an embodiment of a pulse oximetry system 90 that may be configured to implement embodiments of the present disclosure. The system 90 may include a sensor 110, which may be any suitable pulse oximetry sensor, such as those available from Nellcor Puritan Bennett LLC. Light from emitter 16 may pass into a blood perfused tissue, and may be scattered, and then detected by detector 18. A sensor 110 containing an emitter 16 and a detector 18 may also contain an encoder 116 which may be capable of providing signals indicative of the wavelength(s) of light source 16 to allow the oximeter to select appropriate calibration coefficients for calculating oxygen saturation. The encoder 116 may, in an embodiment, be a resistor.

In embodiments, the sensor assembly 110 may be coupled to a cable that is responsible for transmitting electrical and/or optical signals to and from the emitter 16 and detector 18 of the sensor assembly 110. The cable may be permanently coupled to the sensor 110, or it may be removably coupled to the sensor 110—the latter alternative being more useful and cost efficient in situations where the sensor 110 is disposable. In an embodiment, such a device may include a code or other identification parameter that may allow the monitor 100 to select an appropriate software or hardware instruction for processing the signal. In an embodiment of a two-wavelength system, the particular set of coefficients chosen for any pair of wavelength spectra may be determined by a value indicated by the encoder 116 corresponding to a particular light source in a particular sensor 110. In one embodiment, multiple resistor values may be assigned to select different sets of coefficients. In another embodiment, the same resistors are used to select from among the coefficients appropriate for an infrared source paired with either a near red source or far red source. The selection between whether the near red or far red set will be chosen can be selected with a control input from control inputs 154. Control inputs 154 may be, for instance, a switch on the pulse oximeter, a keyboard, or a port providing instructions from a remote host computer. Furthermore, any number of methods or algorithms may be used to determine a patient's pulse rate, oxygen saturation or any other desired physiological parameter.

In an embodiment, the sensor 110 may be connected to a pulse oximetry monitor 100. Monitor 100 may be any standalone or multiparameter monitor, such as one that includes a gel battery 156. The gel battery 156 may be a flexible or inflexible gel battery, and in certain embodiments, it may also be suitable to use a standard cell gel battery, which may also provide quick recharging times. The monitor 100 may also include functionality to use an AC power source for standard power consumption and/or battery recharging, and a switch to use the gel battery 156 when an AC power source is not available.

The monitor 100 may include processing capabilities for determining oxygen saturation and/or heart rate. For example, the monitor 100 may include a microprocessor 122, such as a general-purpose or special-purpose processor, coupled to an internal bus 124. Also connected to the bus may be a RAM memory 126 and a display 128. A time processing unit (TPU) 130 may provide timing control signals to light drive circuitry 132, which controls when the emitter 16 is activated, and if multiple light sources are used, the multiplexed timing for the different light sources. TPU 130 may also control the gating-in of signals from detector 18 through an amplifier 133 and a switching circuit 134. These signals are sampled at the proper time, depending at least in part upon which of multiple light sources is activated, if multiple light sources are used. The received signal from the detector 18 may be passed through an amplifier 136, a low pass filter 138, and/or an analog-to-digital converter 140. The digital data may then be stored in a queued serial module (QSM) 142, for later downloading to RAM 126 as QSM 142 fills up. The monitor 100 may display the calculated patient parameter information on display 128, which may be an electronic paper display.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. Rather, the various embodiments may to cover all modifications, equivalents, and alternatives falling within the spirit and scope of the disclosure as defined by the following appended claims

What is claimed is:

1. A wearable sensor assembly comprising:
a structure capable of being applied to a patient's tissue;
a medical sensor disposed on the structure, wherein the medical sensor is capable of providing a signal related to a patient parameter;
a processor coupled to the medical sensor, wherein the processor is capable of receiving and processing the signal related to the patient parameter to provide an output related to the patient parameter; and
an electronic paper display coupled to the processor, wherein the electronic paper display is capable of displaying the output related to the patient parameter.

2. The wearable sensor assembly, as set forth in claim 1, comprising a flexible gel battery capable of providing power to one or more of the medical sensor, the processor, or the electronic paper display.

3. The wearable sensor assembly, as set forth in claim 2, wherein the flexible gel battery is capable of being recharged in less than one minute.

4. The wearable sensor assembly, as set forth in claim 1, wherein the structure comprises a wristband, a headband, or a hat.

5. The wearable sensor assembly, as set forth in claim 1, wherein the medical sensor comprises an emitter and a detector.

6. The wearable sensor assembly, as set forth in claim 1, wherein the medical sensor comprises a pulse oximetry sensor.

7. The wearable sensor assembly, as set forth in claim 1, wherein the processor output comprises an oxygen saturation value.

8. The wearable sensor assembly, as set forth in claim 1, wherein the processor output comprises a heart rate value.

9. The wearable sensor assembly, as set forth in claim 1, wherein the electronic paper display comprises an electrophoretic display, an electronic ink display, an electro-wetting display, a bistable liquid crystal display, or a cholesteric liquid crystal display.

10. The wearable sensor assembly, as set forth in claim 1, wherein the electronic paper display consumes power only upon updating the display.

11. The wearable sensor assembly, as set forth in claim 1, wherein the display updates once a second or less frequently.

12. A pulse oximetry system comprising:
a pulse oximetry sensor, the pulse oximetry sensor comprising:
an emitter capable of shining light through a patient's tissue; and
a detector capable of detecting the light and providing a signal related to a patient parameter;
a pulse oximetry monitor comprising a processor coupled to the pulse oximetry sensor, wherein the processor is capable of receiving and processing the signal related to the patient parameter to provide an output related to the patient parameter;
a display coupled to the processor, wherein the display is capable of displaying the output related to the patient parameter; and
a gel battery capable of providing power to one or more of the pulse oximetry sensor, the processor, or the display.

13. The pulse oximetry system, as set forth in claim 12, wherein the gel battery comprises a flexible gel battery.

14. The pulse oximetry system, as set forth in claim 12, wherein the gel battery is capable of being recharged in less than 30 seconds.

15. The pulse oximetry system, as set forth in claim 12, wherein the processor output comprises an oxygen saturation value.

16. The pulse oximetry system, as set forth in claim 12, wherein the processor output comprises a heart rate value.

17. The pulse oximetry system, as set forth in claim 12, wherein the display comprises an electronic paper display comprising one or more of an electrophoretic display, an electronic ink display, an electro-wetting display, a bistable liquid crystal display, or a cholesteric liquid crystal display.

18. The pulse oximetry system, as set forth in claim 12, wherein the electronic paper display consumes power only upon updating the display.

19. The pulse oximetry system, as set forth in claim 12, wherein the display updates once a second or less frequently.

20. A wearable sensor assembly comprising:
a structure capable of being applied to a patient's tissue;
a medical sensor disposed on the structure, wherein the medical sensor is capable of providing a signal related to a patient parameter;
a processor coupled to the medical sensor, wherein the processor is capable of receiving and processing the signal related to the patient parameter to provide an output related to the patient parameter;
an display coupled to the processor, wherein the display is capable of displaying the output related to the patient parameter; and
a gel battery capable of providing power to the medical sensor, the processor, and the display.
